(19) United States
(12) Patent Application Publication
(10) Pub. No.: US 2026/0062458 A1
(43) Pub. Date: Mar. 5, 2026

(54) CHIMERIC ANTIGEN RECEPTOR

(71) Applicant: AUTOLUS LIMITED, London (GB)

(72) Inventors: Martin Pulé, London (GB); Kwee Yong, London (GB); Lydia Lee, London (GB); Ben Draper, London (GB)

(21) Appl. No.: 19/172,331

(22) Filed: Apr. 7, 2025

Related U.S. Application Data
(63) Continuation of application No. 18/321,400, filed on May 22, 2023, now abandoned.
Application No. 18/321,400 is a continuation of application No. 17/077,782, filed on Oct. 22, 2020, now abandoned.
Application No. 17/077,782 is a division of application No. 16/162,747, filed on Oct. 17, 2018, now Pat. No. 10,919,951.

(51) Int. Cl. C07K 14/705; A61K 40/11; A61K 40/31; A61K 40/42; A61K 48/00; C07K 14/725; C07K 16/18; C12N 5/0783; C12N 15/85

(52) U.S. Cl. CPC C07K 14/70578; A61K 40/11; A61K 40/31; A61K 40/4215; C07K 14/7051; C07K 14/70575; C07K 16/18; C12N 5/0646; C12N 15/85; A61K 48/00; A61K 2239/31; A61K 2239/38; A61K 2239/46

(57) ABSTRACT

The present invention provides a chimeric antigen receptor (CAR) comprising: (i) a B cell maturation antigen (BCMA)-binding domain which comprises at least part of a proliferation-inducing ligand (APRIL); (ii) a spacer domain (iii) a transmembrane domain; and (iv) an intracellular T cell signaling domain. The invention also provides the use of such a T-cell expressing such a CAR in the treatment of plasma-cell mediated diseases, such as multiple myeloma.

### CROSS-REFERENCE TO RELATED APPLICATIONS

This is a Continuation of U.S. application Ser. No. 18/321,400, filed May 22, 2023, which is a Continuation of U.S. application Ser. No. 17/077,782, filed Oct. 22, 2020, which is a Divisional of U.S. application Ser. No. 16/162,747, filed Oct. 17, 2018, now U.S. Pat. No. 10,919,951, issued Feb. 16, 2021, which is a Continuation of U.S. application Ser. No. 15/028,064, filed Oct. 11, 2017, now U.S. Pat. No. 10,160,794, issued Dec. 25, 2018, which is a U.S. National Phase of International Application No. PCT/GB2014/53058, filed Oct. 10, 2014, which claims priority to Great Britain Application No. 1317929.6, filed Oct. 10, 2013.

### INCORPORATION BY REFERENCE OF MATERIAL SUBMITTED ELECTRONICALLY

A Sequence Listing is incorporated herein by reference as part of the disclosure. The sequence listing was submitted as a text file named “49342D_Seqlisting·XML”, which was created on May 18, 2023 and is 38,647 bytes in size.

### FIELD OF THE INVENTION

The present invention relates to chimeric antigen receptor (CAR) which binds the B cell maturation antigen (BCMA). T cells expressing such a CAR are useful in the treatment of plasma cell diseases such as multiple myeloma.

### BACKGROUND TO THE INVENTION

### Multiple Myeloma

Multiple Myeloma (myeloma) is a bone-marrow malignancy of plasma cells. Collections of abnormal plasma cells accumulate in the bone marrow, where they interfere with the production of normal blood cells. Myeloma is the second most common hematological malignancy in the U.S. (after non-Hodgkin lymphoma), and constitutes 13% of haematologic malignancies and 1% of all cancers. The disease is burdensome in terms of suffering as well as medical expenditure since it causes pathological fractures, susceptibility to infection, renal and then bone-marrow failure before death.

Unlike many lymphomas, myeloma is currently incurable. Standard chemotherapy agents used in lymphoma are largely ineffective for myeloma. In addition, since CD20 expression is lost in plasma cells, Rituximab cannot be used against this disease. New agents such as Bortezamib and Lenolidomide are partially effective, but fail to lead to long-lasting remissions.

There is thus a need for alternative agents for the treatment of myeloma which have increased efficacy and improved long-term effects.

### Chimeric Antigen Receptors (CARs)

Chimeric antigen receptors are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 3).

The most common form of these molecules use single-chain variable fragments (scFv) derived from monoclonal antibodies to recognize a target antigen. The scFv is fused via a spacer and a transmembrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers. Carpenter et al (2013, Clin Cancer Res 19 (8) 2048-60) describe a CAR which incorporates a scFv against the B-cell maturation antigen (BCMA).

BCMA is a transmembrane protein that is preferentially expressed in mature lymphocytes, i.e. memory B cells, plasmablasts and bone marrow plasma cells. BCMA is also expressed on multiple myeloma cells.

Carpenter et al demonstrate that T cells transduced to express the anti-BCMA CAR are capable of specifically killing myeloma cells from a plasmacytoma of a myeloma patient. Although CAR approaches using anti-BCMA antibodies show promise, a particular consideration when targeting this antigen is the particularly low density of BCMA on myeloma cells, in comparison for instance with CD19 on a lymphoma cell. Hence there is a need to increase the sensitivity of target cell recognition of an anti-BCMA CAR T cell.

### DESCRIPTION OF THE FIGURES

FIG. 1—Ligand Specificity and Function Assignment of APRIL and BAFF

B-cell-activating factor (BAFF, TNFSF13B) interacts with BAFF-Receptor (BAFF-R, TNFRSF13C), B-cell membrane antigen (BCMA, TNFRSF17) and transmembrane activator and calcium modulator and cyclophilin ligand interactor (TACI, TNFRSF13B) while A proliferation-inducing ligand (APRIL, TNFSF13) interacts with BCMA, TACI and proteoglycans. BAFF-R activation affects peripheral B-cell survival, while BCMA may affect plasma cell survival. APRIL interaction with proteoglycans involves acidic sulphated glycol-saminoglycan side-chain containing amino-terminus of APRIL.

FIG. 2—Expression Data of BCMA on Myeloma

Myeloma cells from bone marrow samples from 39 multiple myeloma patients were isolated by a CD138+ magnetic bead selection. These cells were stained with the anti-BCMA monoclonal antibody J6MO conjugated with PE (GSK). Antigen copy number was quantified using PE Quantibrite beads (Becton Dickenson) as per the manufacturer's instructions. A box and whiskers plot of antigen copy number is presented along with the range, interquartile and median values plotted. We found the range is 348.7-4268.4 BCMA copies per cell with a mean of 1181 and a median of 1084.9.

FIG. 3—Standard Design of a Chimeric Antigen Receptor

The typical format of a chimeric antigen receptor is shown. These are type I transmembrane proteins. An ectodomain recognizes antigen. This is composed of an antibody derived single-chain variable fragment (scFv) which is attached to a spacer domain. This in turn is connected to a transmembrane domain which acts to anchor the molecule in the membrane. Finally, this is connected to an endodomain which acts to transmits intracellular signals to the cell. This is composed of one or more signalling domains.

FIGS. 4A-4C—Design of the Different APRIL-Based CARs Generated.

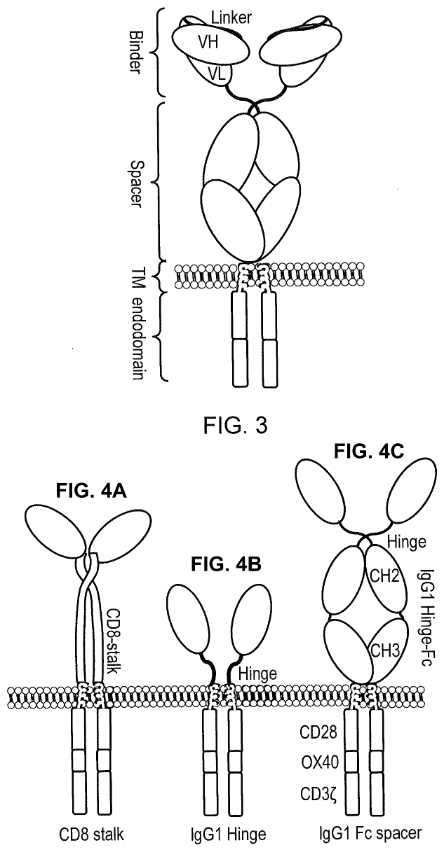

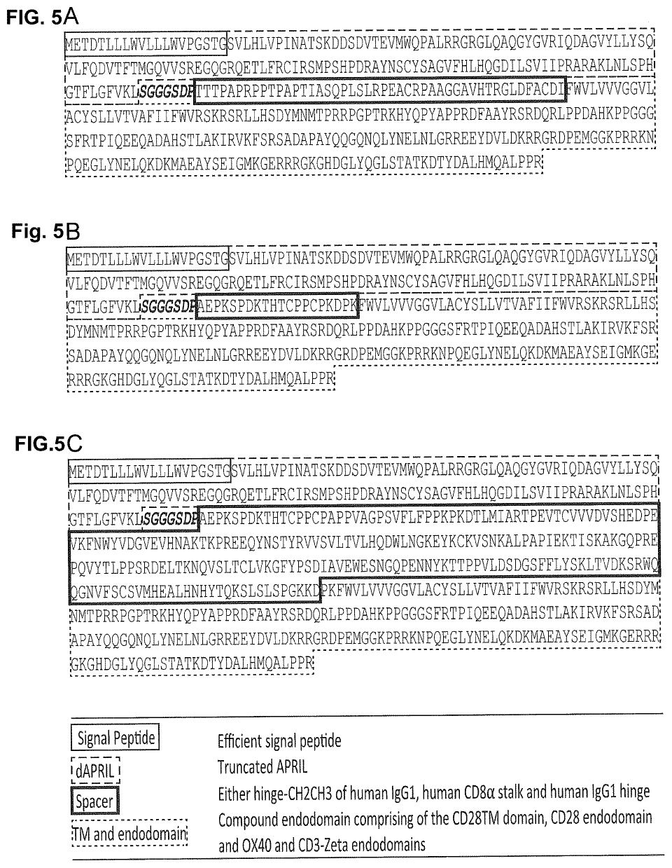

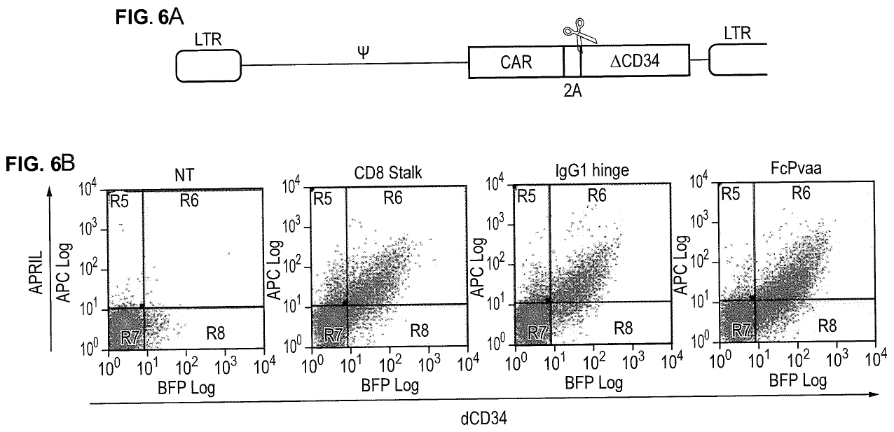

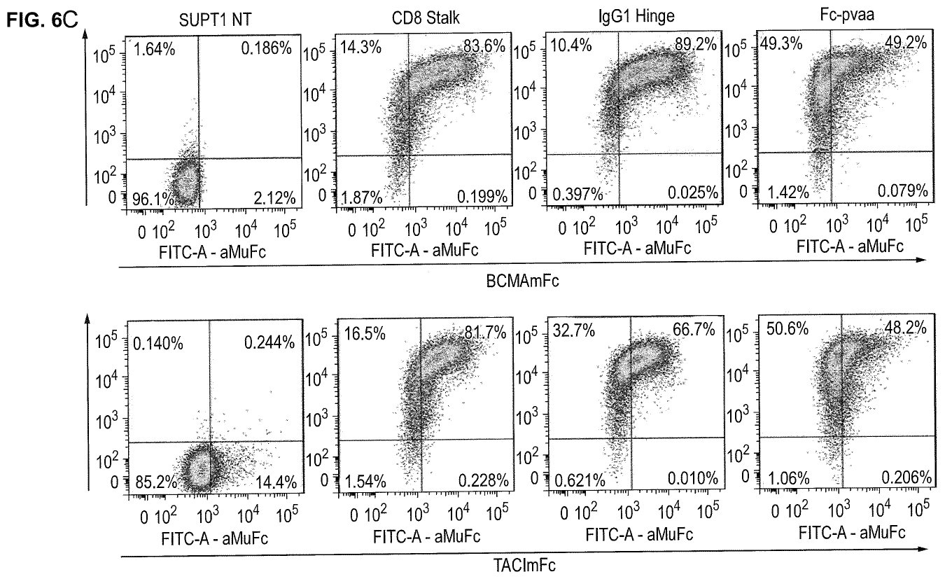

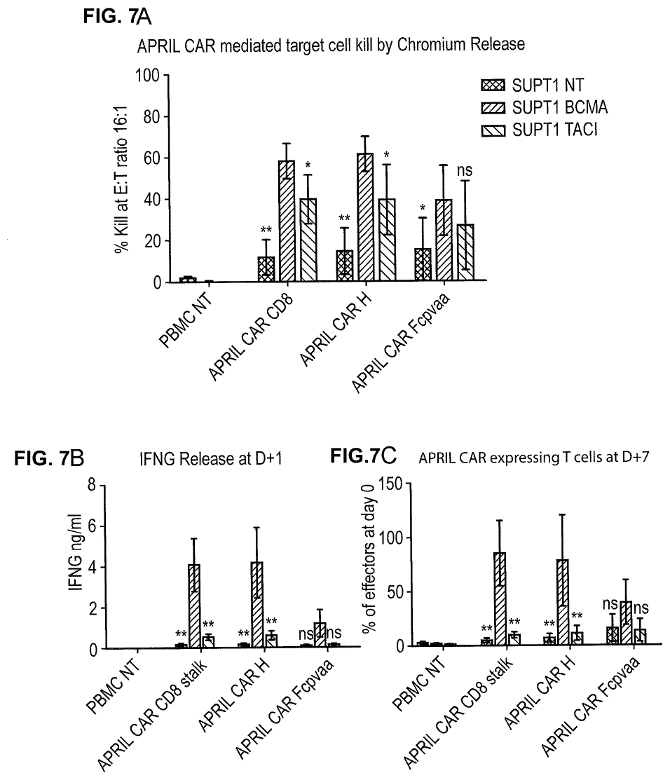

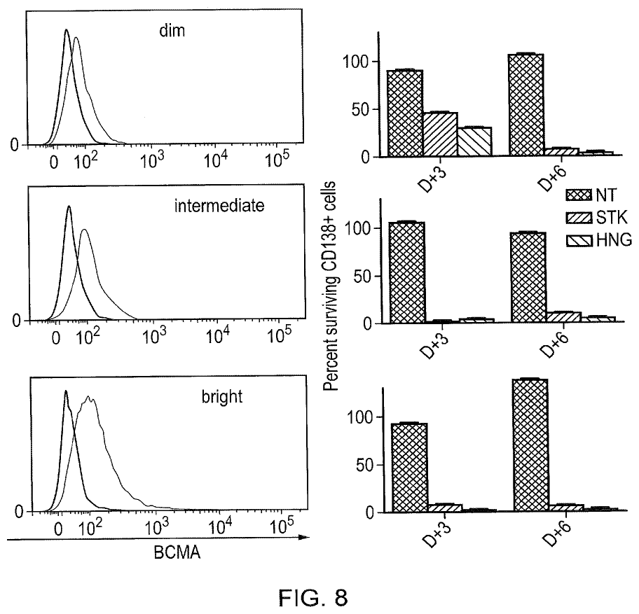

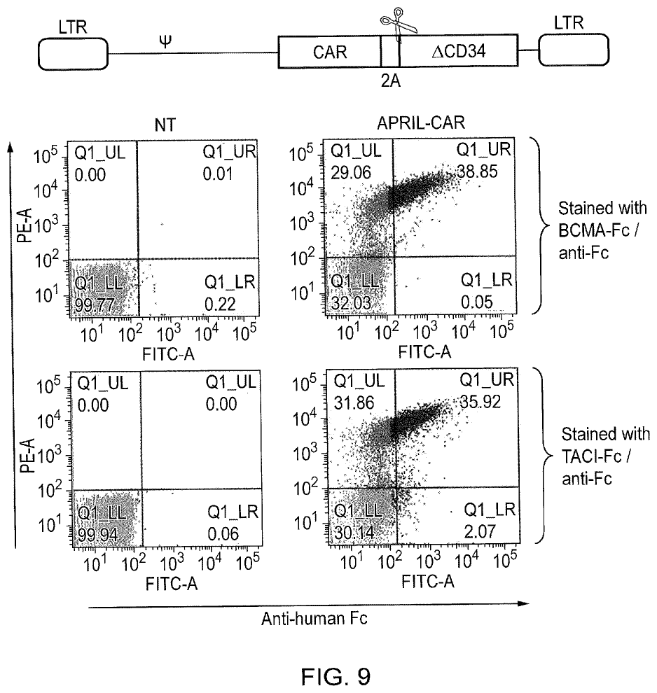

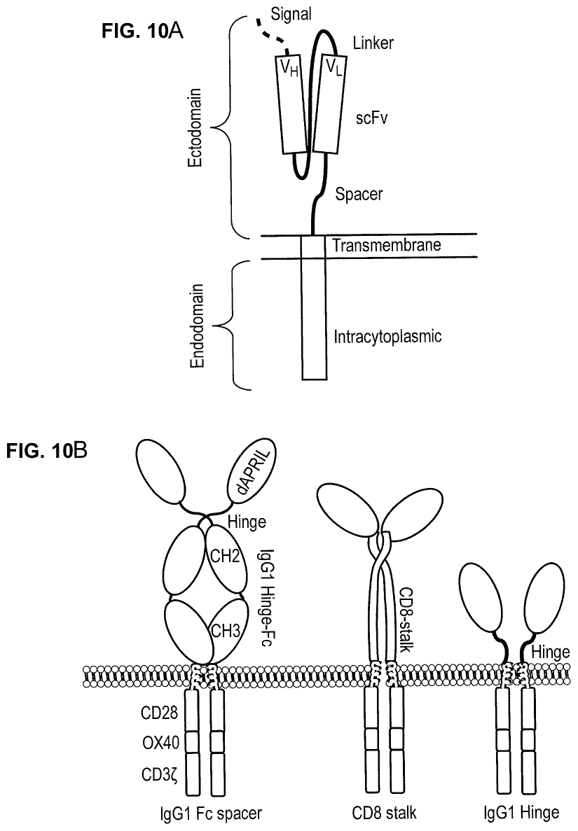

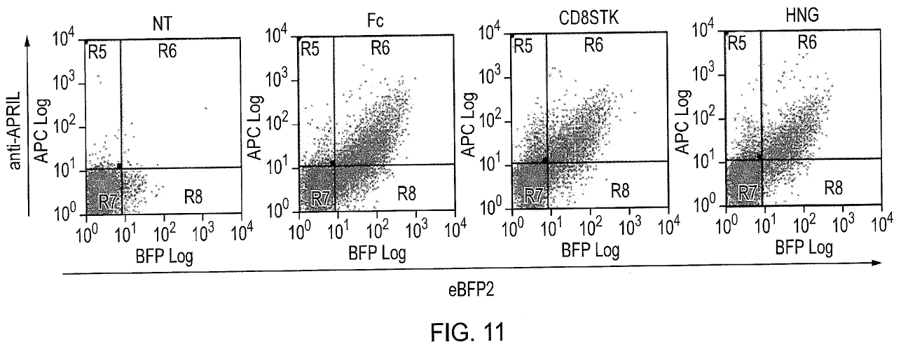

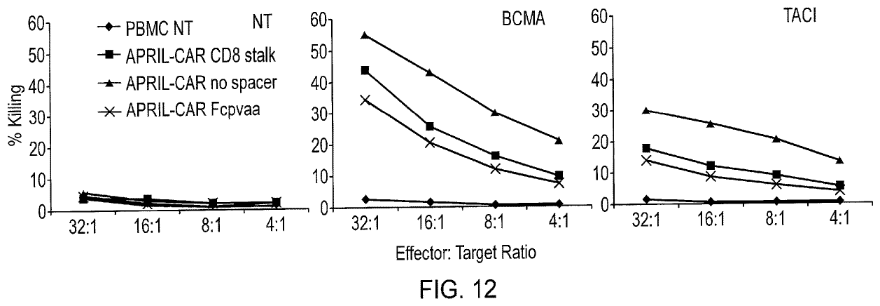

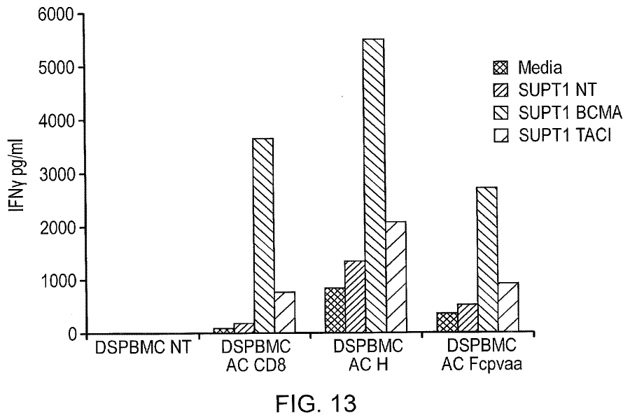

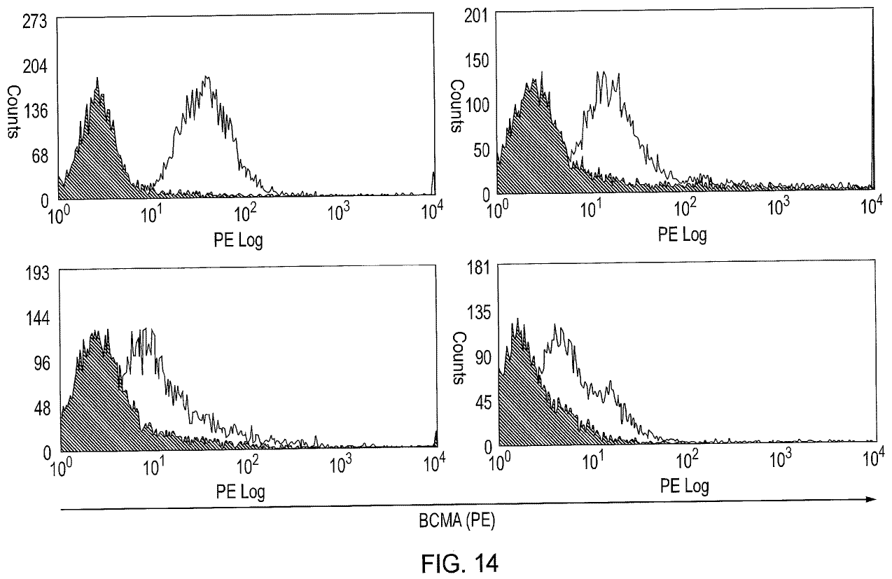

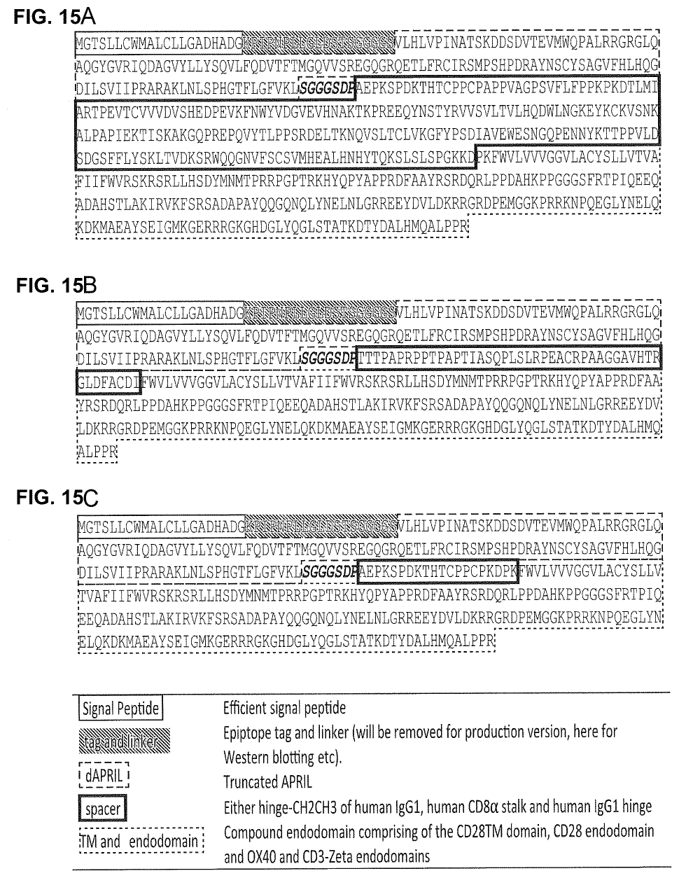

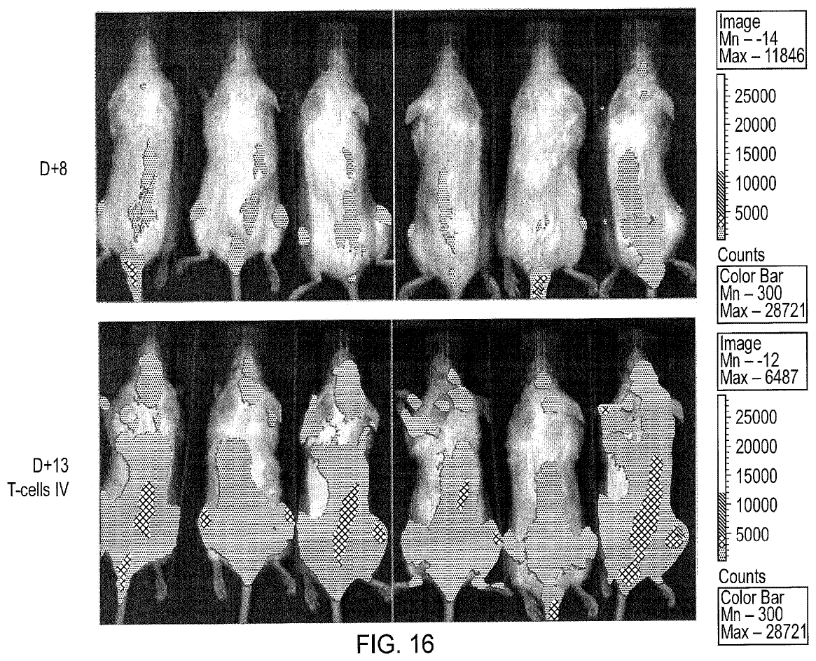

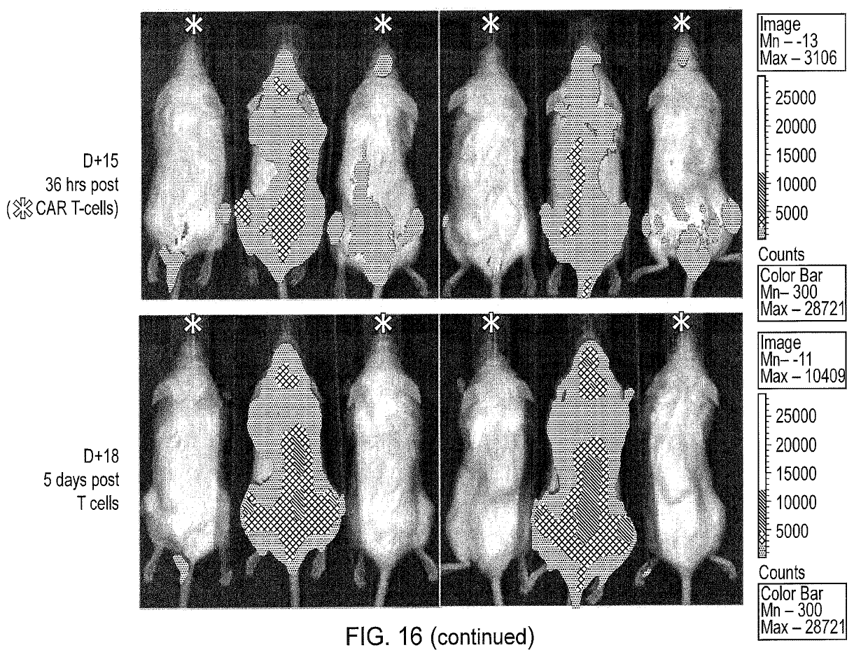

The CAR design as shown in FIG. 3 was modified so that the scFv was replaced with a modified form of APRIL to act as an antigen binding domain: APRIL was truncated so that the proteoglycan binding amino-terminus is absent. A signal peptide was then attached to truncated APRIL amino-terminus to direct the protein to the cell surface. Three CARs were generated with this APRIL based binding domain: FIG. 4A. In the first CAR, the human CD8 stalk domain was used as a spacer domain. FIG. 4B. In the second CAR, the hinge from IgG1 was used as a spacer domain. FIG. 4C. In the third CAR, the hinge, CH2 and CH3 domains of human IgG1 modified with the pva/a mutations described by Hombach et al (2010 Gene Ther. 17:1206-1213) to reduce Fc Receptor binding was used as a spacer (henceforth referred as Fc-pvaa). In all CARS, these spacers were connected to the CD28 transmembrane domain and then to a tripartite endodomain containing a fusion of the CD28, OX40 and the CD3-Zeta endodomain (Pule et al, Molecular therapy, 2005: Volume 12; Issue 5; Pages 933-41).

FIGS. 5A-5C—Annotated Amino Acid Sequence of the Above Three APRIL-CARS

FIG. 5A: Shows the annotated amino acid sequence of the CD8 stalk APRIL CAR; FIG. 5B: Shows the annotated amino acid sequence of the APRIL IgG1 hinge based CAR;

FIG. 5C: Shows the Annotated Amino Acid Sequence of the APRIL Fc-pvaa Based CAR.

FIGS. 6A-6C—Expression and ligand binding of different APRIL based CARs FIG. 6A. The receptors were co-expressed with a marker gene truncated CD34 in a retroviral gene vector. Expression of the marker gene on transduced cells allows confirmation of transduction. FIG. 6B. T-cells were transduced with APRIL based CARs with either the CD8 stalk spacer, IgG1 hinge or Fc spacer. To test whether these receptors could be stably expressed on the cell surface, T-cells were then stained with anti-APRIL-biotin/Streptavidin APC and anti-CD34. Flow-cytometric analysis was performed. APRIL was equally detected on the cell surface in the three CARs suggesting they are equally stably expressed. FIG. 6C. Next, the capacity of the CARs to recognize TACI and BCMA was determined. The transduced T-cells were stained with either recombinant BCMA or TACI fused to mouse IgG2a Fc fusion along with an anti-mouse secondary and anti-CD34. All three receptor formats showed binding to both BCMA and TACI. A surprising finding was that binding to BCMA seemed greater than to TACI. A further surprising finding was that although all three CARs were equally expressed, the CD8 stalk and IgG1 hinge CARs appeared better at recognizing BCMA and TACI than that with the Fc spacer.

FIGS. 7A-7C—Function of the Different Car Constructs.

Functional assays were performed of the three different APRIL based CARs. Normal donor peripheral blood T-cells either non-transduced (NT), or transduced to express the different CARs. Transduction was performed using equal titer supernatant. These T-cells were then CD56 depleted to remove non-specific NK activity and used as effectors. SupT1 cells either non-transduced (NT), or transduced to express BCMA or TACI were used as targets. Data shown is mean and standard deviation from 5 independent experiments. FIG. 7A. Specific killing of BCMA and TACI expressing T-cells was determined using Chromium release. FIG. 7B. Interferon-γ release was also determined. Targets and effectors were co-cultured at a ratio of 1:1. After 24 hours, Interferon-γ in the supernatant was assayed by ELISA. FIG. 7C. Proliferation/survival of CAR T-cells were also determined by counting number of CAR T-cells in the same co-culture incubated for a further 6 days. All 3 CARs direct responses against BCMA and TACI expressing targets. The responses to BCMA were greater than for TACI.

FIG. 8—Killing of Primary Myeloma Cells by April Car T-Cells

Since most primary myeloma cells express a low number of BCMA molecules on their surface, it was investigated whether killing of primary myeloma cells occurs despite low-density expression. Three cases were selected which represented the range of BCMA expression described in FIG. 2: the first had dim expression (lower than mean); the second case had intermediate expression (approximately mean expression) and the third had bright (above mean expression). A histogram of BCMA staining against isotype control for all three cases is shown on the left. In this assay, only the CD8 stalk and hinge APRIL CARs were tested. On the left, survival of myeloma cells compared with starting numbers is shown at day 3 and day 6 after a 1:1 co-culture of myeloma cells and CAR T-cells. By day 6, >95% of the myeloma cells were eliminated, including those with dim BCMA expression.

FIG. 9—Vector Co-Expressing APRIL Based CAR with Truncated CD34

A cell line expressing the vector used for screening was incubated with either BCMA-Fc or TACI-Fc and stained with both anti-CD34 and anti-human-Fc PE and FITC conjugated mAbs. The cells were then studied by flow-cytometery. This shows a typical pattern of binding of BCMA and TACI relative to the marker gene CD34.

FIGS. 10A-10B—FIG. 10A Schematic Diagram Illustrating a Classical CAR

FIG. 10B Design of the Different APRIL-Based CARs Generated.

A signal peptide is attached to truncated APRIL amino-terminus. This was fused to different spacers: either the hinge, CH2 and CH3 domains of human IgG1 modified with the pvaa mutation described by Hombach et al (2010 Gene Ther. 17:1206-1213) to reduce Fc Receptor binding; the stalk of human CD8α; and the hinge of IgG1. These spacers were connected to a tripartite endodomain containing CD28 transmembrane domain, the OX40 endodomain and the CD3-Zeta endodomain.

FIG. 11—Expression of Different CARS

The receptors were co-expressed with enhanced blue fluorescence protein 2 (eBFP2) using an IRES sequence. Primary human T-cells were transduced and stained with anti-APRIL-biotin/Streptavidin APC. Flow-cytometric analysis was performed. eBFP2 signal is shown against APRIL detection. All three CARs are stably expressed (representative experiment of 3 independent experiments performed using 3 different normal donor T-cells).

FIG. 12—Chromium Release Assay

Using normal donor peripheral blood T-cells either non-transduced (NT), or transduced to express different spacer CARs as effectors, and SupT1 cells either non-transduced (NT), or transduced to express BCMA or TACI as targets. The T-cells were CD56 depleted to reduce NK activity. This is a representative of three independent experiments and is shown as an example. Cumulative killing data is shown in FIG. 7A. Specific killing of BCMA and TACI expressing T-cells is seen with no activity against negative target cells.

FIG. 13—Interferon-Gamma Release

From a 1:1 co-culture of effectors and targets is measured by ELISA. The CD8 stalk construct appears to have the best specificity while the hinge construct results in the most Interferon release demonstrates some non-specific activity. This is representative of 3 independent experiments and is shown as an example. Cumulative interferon-gamma release data is shown in FIG. 7B.

FIG. 14—Examples of BCMA Expression on Primary Myelomas

Four examples of myeloma samples stained with the rat anti-human BCMA mAb Vickyl is shown. The first panel shows bright BCMA staining in a patient with a plasma cell leukemia (an unusual, advanced and aggressive form of myeloma). The other three cases are clinically and morphologically typical myelomas. They show the intermediate or dim staining typically seen. Staining with isotype control (grey) is superimposed. These are examples of cumulative BCMA expression data shown in FIG. 2.

FIGS. 15A-15C—Amino Acid Sequence of APRIL-CARS with a V5 Epitope Tag.

FIG. 15A: dAPRIL-HCH2CH3pvaa-CD28OXZ

FIG. 15B: dAPRIL-CD8STK-CD28OXZ

FIG. 15C: dAPRIL-HNG-CD28OXZ

Sequences in this figure differ from those in FIG. 5 have a different signal peptide and no V5 tag.

FIG. 16—Demonstration of In Vivo Function of APRIL CAR T-Cells

Six 3 month old female NSG mice received 1×10⁷ MM1·s·FLuc cells vial tail-vein injection. Mice were imaged with bioluminescence at day 8 and day 13. After imaging on day 13, four mice received 5×10⁶ APRIL CAR T-cells via tail vein injection. Mice were imaged on day 13 and day 18. Mice which received CAR T-cells are indicated with (*). Remission of Myeloma could be observed by Day 18 in all treated mice, while disease in untreated mice progressed.

### SUMMARY OF ASPECTS OF THE INVENTION

B-cell membrane antigen (BCMA) is a surface protein expressed on nearly all Multiple Myeloma (MM). BCMA is only otherwise expressed on plasma cells hence targeting this antigen may prove an effective treatment of myeloma. However, the low-level expression of BCMA (See FIG. 2), is a consideration when targeting this antigen.

The present inventors have surprisingly found that if a binding domain is used based on A proliferation-inducing ligand (APRIL), rather than a BCMA-binding antibody, in a CAR-type molecule, T cells expressing such CARs cause very efficient killing of BCMA-expressing target cells, even those with low-levels of expression.

Without wishing to be bound by theory, the present inventors predict that this is because the three-fold symmetry inherent in the binding of BCMA with APRIL. This means that every interaction between the CAR and BCMA will involve 3 CARS, approximating 3 endodomains on the T-cell surface. Since T-cell activation is triggered by close approximation of signalling endodomains in an immunological synapse, the CAR design of the present invention is highly sensitive and specific. As BCMA is expressed at a very low density on primary myeloma cells (see FIGS. 2 and 7), this receptor design is particularly suited to this target.

Thus, in a first aspect the present invention provides a chimeric antigen receptor (CAR) comprising:
(i) a B cell maturation antigen (BCMA)-binding domain which comprises at least part of a proliferation-inducing ligand (APRIL);
(ii) a spacer domain
(iii) a transmembrane domain; and
(iv) an intracellular T cell signaling domain.

The BCMA-binding domain may comprise a truncated APRIL which comprises the BCMA binding site but lacks the amino terminal portion of APRIL responsible for proteoglycan binding. Such a molecule may comprise the sequence shown as SEQ ID No. 14. Alternatively the molecule may comprise a variant of that sequence having at least 80% sequence identity which binds BCMA.

The transmembrane and intracellular T-cell signalling domain may comprise the sequence shown as SEQ ID No. 7 or a variant thereof having at least 80% sequence identity.

The BCMA-binding domain and the transmembrane domain may be connected by a spacer. The spacer may comprise one of the following: a human IgG1 spacer; an IgG1 hinge; or a CD8 stalk.

The CAR of the first aspect of the invention may comprise the sequence shown as SEQ ID No. 1, 2, 3, 4, 5 or 6 or a variant thereof which has at least 80% sequence identity but retains the capacity to i) bind BCMA and ii) induce T cell signalling.

The CAR of the first aspect of the invention may bind to BCMA as a trimer.

In a second aspect, the present invention provides a nucleic acid sequence which encodes a CAR according to any preceding claim.

The nucleic acid sequence may comprise the sequence shown as SEQ ID No 15, 16, 17, 18, 19 or 20 or a variant thereof having at least 80% sequence identity.

In a third aspect, the present invention provides a vector which comprises a nucleic acid sequence according to the second aspect of the invention.

In a fourth aspect, the present invention provides a T cell or an NK cell which expresses a CAR according to the first aspect of the invention.

In a fifth aspect, the present invention provides a method for making a T cell or an NK cell according to the fourth aspect of the invention, which comprises the step of introducing a nucleic acid according to the second aspect of the invention into a T cell or an NK cell.

In a sixth aspect, the present invention provides a pharmaceutical composition which comprises a vector according to the third aspect of the invention or T cell/NK cell according to the fourth aspect of the invention, together with a pharmaceutically acceptable carrier, diluent or excipient.

In a seventh aspect, the present invention provides a method for treating a plasma cell disorder which comprises the step of administering a vector according to the third aspect of the invention or T cell/NK cell according to the fourth aspect of the invention to a subject.

The plasma cell disorder may be selected from plasmacytoma, plasma cell leukemia, multiple macroglobulinemia, myeloma, amyloidosis, Waldenstrom's macroglobulinemia, solitary bone plasmacytoma, extramedullary plasmacytoma, osteosclerotic myeloma, heavy chain diseases, monoclonal gammopathy of undetermined significance and smoldering multiple myeloma.

The plasma cell disorder may be multiple myeloma.

In an eighth aspect, the present invention provides a vector according to the third aspect of the invention or T cell/NK cell according to the fourth aspect of the invention for use in treating a plasma cell disorder.

In a ninth aspect, the present invention provides use of a vector according to the third aspect of the invention or T cell/NK cell according to the fourth aspect of the invention in the manufacture of a medicament for treating a plasma cell disorder.

### DETAILED DESCRIPTION

### Chimeric Antigen Receptors (Cars)

Chimeric antigen receptors (CARs), also known as chimeric T cell receptors, artificial T cell receptors and chimeric immunoreceptors, are engineered receptors, which graft an arbitrary specificity onto an immune effector cell. In a classical CAR (FIG. 3), the specificity of a monoclonal antibody is grafted on to a T cell or NK cell. CAR-encoding nucleic acids may be introduced into T cells or NK cells using, for example, retroviral vectors. In this way, a large number of cancer-specific T cells or NK cells can be generated for adoptive cell transfer. Early clinical studies of this approach have shown efficacy in some cancers, primarily when targeting the pan-B-cell antigen CD19 to treat B-cell malignancies.

The target-antigen binding domain of a CAR is commonly fused via a spacer and transmembrane domain to a signaling endodomain. When the CAR binds the target-antigen, this results in the transmission of an activating signal to the T-cell it is expressed on.

The CAR of the present invention comprises:
(i) a B cell maturation antigen (BCMA)-binding domain which comprises at least part of a proliferation-inducing ligand (APRIL), which is discussed in more detail below;
(ii) a spacer
(iii) a transmembrane domain; and
(iv) an intracellular T cell signaling domain

The CAR of the present invention may comprise one of the following amino acid sequences:

(dAPRIL-HCH2CH3pvaa-CD28OXZ)
SEQ ID No. 1
METDTLLLWVLLLWVPGSTGSVLHLVPINATSKDDSDVTEVMWQPALRRGRGLQAQGYGVRIQ
DAGVYLLYSQVLFQDVTFTMGQVVSREGQGRQETLFRCIRSMPSHPDRAYNSCYSAGVFHLHQ
GDILSVIIPRARAKLNLSPHGTFLGFVKLSGGGSDPAEPKSPDKTHTCPPCPAPPVAGPSVFL
FPPKPKDTLMIARTPEVTCVVVDVSHEDPEVKFNWYVDGVEVHNAKIKPREEQYNSTYRVVSV
LTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHEA
LHNHYTQKSLSLSPGKKDPKFWVLVVVGGVLACYSLLVTVAFIIFWVRSKRSRLLHSDYMNMT
PRRPGPTRKHYQPYAPPRDFAAYRSRDQRLPPDAHKPPGGGSFRTPIQEEQADAHSTLAKIRV
KFSRSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
(dAPRIL-CD8STK-CD28OXZ)
SEQ ID No. 2
METDTLLLWVLLLWVPGSTGSVLHLVPINATSKDDSDVTEVMWQPALRRGRGLQAQGYGVRIQ
DAGVYLLYSQVLFQDVTFTMGQVVSREGQGRQETLFRCIRSMPSHPDRAYNSCYSAGVFHLHQ
GDILSVIIPRARAKLNLSPHGTFLGFVKLSGGGSDPTTTPAPRPPTPAPTIASQPLSLRPEAC
RPAAGGAVHTRGLDFACDIFWVLVVVGGVLACYSLLVTVAFIIFWVRSKRSRLLHSDYMNMTP
RRPGPTRKHYQPYAPPRDFAAYRSRDQRLPPDAHKPPGGGSFRTPIQEEQADAHSTLAKIRVK
FSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
(dAPRIL-HNG-CD28OXZ)
SEQ ID No. 3
METDTLLLWVLLLWVPGSTGSVLHLVPINATSKDDSDVTEVMWQPALRRGRGLQAQGYGVRIQ
DAGVYLLYSQVLFQDVTFTMGQVVSREGQGRQETLFRCIRSMPSHPDRAYNSCYSAGVFHLHQ
GDILSVIIPRARAKLNLSPHGTFLGFVKLSGGGSDPAEPKSPDKTHTCPPCPKDPKFWVLVVV
GGVLACYSLLVTVAFIIFWVRSKRSRLLHSDYMNMTPRRPGPTRKHYQPYAPPRDFAAYRSRD
QRLPPDAHKPPGGGSFRTPIQEEQADAHSTLAKIRVKFSRSADAPAYQQGQNQLYNELNLGRR
EEYDVLDKRRGRDPEMGGKPRRKNPQEGLYNELQKDKMAEAYSEIGMKGERRRGKGHDGLYQG
LSTATKDTYDALHMQALPPR
(dAPRIL-HCH2CH3pvaa-CD28OXZ)
SEQ ID No. 4
MGTSLLCWMALCLLGADHADGKPIPNPLLGLDSTSGGGGSVLHLVPINATSKDDSDVTEVMWQ
PALRRGRGLQAQGYGVRIQDAGVYLLYSQVLFQDVTFTMGQVVSREGQGRQETLFRCIRSMPS
HPDRAYNSCYSAGVFHLHQGDILSVIIPRARAKLNLSPHGTFLGFVKLSGGGSDPAEPKSPDK
THTCPPCPAPPVAGPSVFLFPPKPKDTLMIARTPEVTCVVVDVSHEDPEVKFNWYVDGVEVHN
AKTKPREEQYNSTYRVVSVLIVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGKKDPKFWVLVVVGGVLACYSLLVTVAFII
FWVRSKRSRLLHSDYMNMTPRRPGPTRKHYQPYAPPRDFAAYRSRDQRLPPDAHKPPGGGSFR
TPIQEEQADAHSTLAKIRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTYDALHMQAL
PPR
(dAPRIL-CD8STK-CD28OXZ)
SEQ ID No. 5
MGTSLLCWMALCLLGADHADGKPIPNPLLGLDSTSGGGGSVLHLVPINATSKDDSDVTEVMWQ
PALRRGRGLQAQGYGVRIQDAGVYLLYSQVLFQDVTFTMGQVVSREGQGRQETLFRCIRSMPS
HPDRAYNSCYSAGVFHLHQGDILSVIIPRARAKLNLSPHGTFLGFVKLSGGGSDPTTTPAPRP
PTPAPTIASQPLSLRPEACRPAAGGAVHTRGLDFACDIFWVLVVVGGVLACYSLLVTVAFIIF
WVRSKRSRLLHSDYMNMTPRRPGPTRKHYQPYAPPRDFAAYRSRDQRLPPDAHKPPGGGSFRT
PIQEEQADAHSTLAKIRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHMQALP
PR
(dAPRIL-HNG-CD28OXZ)
SEQ ID No. 6
MGTSLLCWMALCLLGADHADGKPIPNPLLGLDSTSGGGGSVLHLVPINATSKDDSDVTEVMWQ
PALRRGRGLQAQGYGVRIQDAGVYLLYSQVLFQDVTFTMGQVVSREGQGRQETLFRCIRSMPS
HPDRAYNSCYSAGVFHLHQGDILSVIIPRARAKLNLSPHGTFLGFVKLSGGGSDPAEPKSPDK
THTCPPCPKDPKFWVLVVVGGVLACYSLLVTVAFIIFWVRSKRSRLLHSDYMNMTPRRPGPTR
KHYQPYAPPRDFAAYRSRDQRLPPDAHKPPGGGSFRTPIQEEQADAHSTLAKIRVKFSRSADA
PAYQQGQNQLYNELNLGRREEYDVLDKRRGRDPEMGGKPRRKNPQEGLYNELQKDKMAEAYSE
IGMKGERRRGKGHDGLYQGLSTATKDTYDALHMQALPPR

The molecule of the invention may comprise a variant of the sequence shown as SEQ ID No. 1, 2, 3, 4, 5 or 6 having at least 80, 85, 90, 95, 98 or 99% sequence identity, provided that the variant sequence is a molecule as defined in the first aspect of the invention, i.e. a CAR which comprises:
(i) a BCMA-binding domain;
(ii) a spacer domain
(iii) a transmembrane domain; and
(iv) an intracellular T cell signaling domain.

The percentage identity between two polypeptide sequences may be readily determined by programs such as BLAST which is freely available at http://blast.ncbi.nlm.nih.gov.

### Transmembrane Domain

The transmembrane domain is the sequence of the CAR that spans the membrane. It may comprise a hydrophobic alpha helix. The transmembrane domain may be derived from CD28, which gives good receptor stability. The transmembrane domain may be derived from any type I transmembrane protein. The transmembrane domain may be a synthetic sequence predicted to form a hydrophobic helix.

### Intracellular T Cell Signaling Domain (Endodomain)

The endodomain is the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together (Pule et al, Molecular therapy, 2005: Volume 12; Issue 5; Pages 933-41). The CAR endodomain may also be derived from other signaling domains either individually or in combination, derived from signaling proteins found in nature or artificial ones constructed by those skilled in the art such that the CAR transmits a suitable signal to for an effective CAR therapeutic.

The endodomain of the CAR of the present invention may comprise the CD28 endodomain and OX40 and CD3-Zeta endodomain.

The transmembrane and intracellular T-cell signalling domain (endodomain) of the CAR of the present invention may comprise the sequence shown as SEQ ID No. 7 or a variant thereof having at least 80% sequence identity.

SEQ ID No. 7
FWVLVVVGGVLACYSLLVTVAFIIFWVRSKRSRLLHSDYMNMTPRRP
GPTRKHYQPYAPPRDFAAYRSRDQRLPPDAHKPPGGGSFRTPIQEEQ
ADAHSTLAKIRVKFSRSADAPAYQQGQNQLYNELNLGRREEYDVLDK
RRGRDPEMGGKPRRKNPQEGLYNELQKDKMAEAYSEIGMKGERRRGK
GHDGLYQGLSTATKDTYDALHMQALPPR

A variant sequence may have at least 80%, 85%, 90%, 95%, 98% or 99% sequence identity to SEQ ID No. 7, provided that the sequence provides an effective transmembrane domain and an effective intracellular T cell signaling domain.

### Signal Peptide

The CAR of the present invention may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The CAR of the invention may have the general formula:

Signal peptide-BCMA-binding domain-spacer domain-transmembrane domain-intracellular T cell signaling domain.

The signal peptide may comprise the SEQ ID No. 8 or 9 or a variant thereof having 5, 4, 3, 2 or 1 amino acid mutations (insertions, substitutions or additions) provided that the signal peptide still functions to cause cell surface expression of the CAR.

SEQ ID No. 8:
MGTSLLCWMALCLLGADHADG
SEQ ID No. 9:
METDTLLLWVLLLWVPGSTG

The signal peptide of SEQ ID No. 8 and SEQ ID No 9 is compact and highly efficient. It is predicted to give about 95% cleavage after the terminal glycine, giving efficient removal by signal peptidase.

### Spacer

The CAR of the present invention may comprise a spacer sequence to connect the BCMA-binding domain with the transmembrane domain and spatially separate the BCMA-binding domain from the endodomain. A flexible spacer allows to the BCMA-binding domain to orient in different directions to enable BCMA binding.

The spacer sequence may, for example, comprise an IgG1 Fc region, an IgG1 hinge or a CD8 stalk. The linker may alternatively comprise an alternative linker sequence which has similar length and/or domain spacing properties as an IgG1 Fc region, an IgG1 hinge or a CD8 stalk.

The spacer may be a short spacer, for example a spacer which comprises less than 100, less than 80, less than 60 or less than 45 amino acids. The spacer may be or comprise an IgG1 hinge or a CD8 stalk or a modified version thereof.

A human IgG1 spacer may be altered to remove Fc binding motifs.

Examples of amino acid sequences for these spacers are given below:

SEQ ID No. 10 (hinge-CH2CH3 of human IgG1)
AEPKSPDKTHTCPPCPAPPVAGPSVFLFPPKPKDTLMIARTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRD
ELTKNQVSLTCLVKGFYPSDIAVEWESNGQPENNYKTTPPVLDSDGS
FFLYSKLTVDKSRWQQGNVFSCSVMHEALHNHYTQKSLSLSPGKKD
SEQ ID No. 11 (human CD8 stalk):
TTTPAPRPPTPAPTIASQPLSLRPEACRPAAGGAVHTRGLDFACDI
SEQ ID No. 12 (human IgG1 hinge):
AEPKSPDKTHTCPPCPKDPK

### B-Cell Membrane Antigen (BCMA)

The CAR of the first aspect of the invention comprises a domain which binds BCMA.

BCMA, also known as TNFRSF17, is a plasma cell specific surface antigen which is expressed exclusively on B-lineage haemopoietic cells or dendritic cells. It is a member of the TNF receptor family. BCMA is not expressed on naïve B cells but is up-regulated during B-cell differentiation into plasmablasts, and is brightly expressed on memory B cells, plasmablasts and bone marrow plasma cells. BCMA is also expressed on the majority of primary myeloma cells. Unlike other CAR targets such as CD19, BCMA is expressed at low density (FIG. 2).

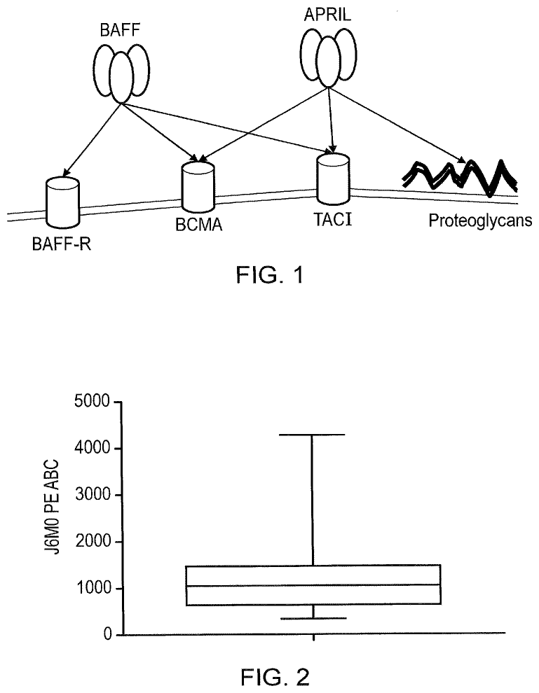

BCMA functions within a network of interconnected ligands and receptors which is shown schematically in FIG. 1. Two other TNF receptors share the ligands APRIL and BAFF with BCMA-TACI (TNFRSF13B), which is found on activated T-cells and all B-cells and BAFF-R (TNFRSF13C) which is predominantly expressed on B-lymphocytes. Multiple myeloma cells express TACI in some cases and BCMA in most cases, but never BAFF-R.

### April

The BCMA-binding domain of the CAR of the invention and comprises at least part of a proliferation-inducing ligand (APRIL). APRIL is also known as TNFSF13.

The wild-type sequence of APRIL is available at UNIPROT/O75888 and is show below (SEQ ID No. 13). It is not a classical secreted protein in that it has no signal peptide. It has a furin cleavage site “KQKKQK” (underlined in SEQ ID No. 13). The amino terminus is involved in proteoglycan binding.

The BCMA-binding domain may comprise the BCMA-binding site of APRIL. The BCMA-binding domain may comprise a fragment of APRIL which comprises the BCMA-binding site.

The BCMA-binding domain may comprise a truncated APRIL, which lacks the amino terminal end of the molecule. The truncated APRIL may retain BCMA and TACI binding but lose proteoglycan binding. Truncated APRIL can be cleaved at or immediately after the furin cleavage site. Truncated APRIL may lack the amino terminal 116 amino acids from the wild-type APRIL molecule shown as SEQ ID No. 13. Truncated APRIL may comprise the sequence shown as SEQ ID No. 14 (which corresponds to the portion of SEQ ID No. 13 shown in bold) or a variant thereof. This corresponds to the portion of the molecule which is needed for BCMA and TACI binding.

```
SEQ ID No. 13
                10 20 30 40
MPASSPFLLA PKGPPGNMGG PVREPALSVA LWLSWGAALG
                50 60 70 80
AVACAMALLT QQTELQSLRR EVSRLQGTGG PSQNGEGYPW
                90 100 110 120
QSLPEQSSDA LEAWENGERS RKRRAVLTQK QKKQHSVLHL
              130 140 160 170
VPINATSKDD SDVTEVMWQP ALRRGRGLQA GVYLLYSQVL
              180 190 200 210
FQDVTFTMGQ VVSREGQGRQ ETLFRCIRSM PSHPDRAYNS
              220 230 240 250
CYSAGVFHLH QGDILSVIIP RARAKLNLSP HGTFLGFVKL
SEQ ID No. 14
VLHLVPINATSKDDSDVTEVMWQPALRRGRGLQAQGYGVRIQDAGVYLLY
SQVLFQDVTFTMGQVVSREGQGRQETLFRCIRSMPSHPDRAYNSCYSAGV
FHLHQGDILSVIIPRARAKLNLSPHGTFLGFVKL
```

The CAR of the present invention may comprise a variant of the truncated APRIL molecule shown as SEQ ID No. 14 which has at least 80% amino acid sequence identity and which has the same or improved BCMA binding capabilities. The variant sequence may have at least 80%, 85%, 90%, 95%, 98% or 99% sequence identity to SEQ ID No. 14.

### Nucleic Acid Sequence

The second aspect of the invention relates to a nucleic acid sequence which codes for a CAR of the first aspect of the invention.

The nucleic acid sequence may be or comprise one of the following sequences:

(dAPRIL-HCH2CH3pvaa-CD28OXZ)
SEQ ID No. 15
ATGGAGACCGACACCCTGCTGCTGTGGGTGCTGCTGCTGTGGGTGCCAGGCAGCACCGGCAGC
GTGCTCCACCTGGTGCCCATCAACGCCACCAGCAAGGACGACTCTGATGTGACCGAGGTGATG
TGGCAGCCAGCCCTGAGACGGGGCAGAGGCCTGCAGGCCCAGGGCTACGGCGTGAGAATCCAG
GACGCTGGCGTGTACCTGCTGTACTCCCAGGTGCTGTTCCAGGACGTGACCTTCACAATGGGC
CAGGTGGTGAGCCGGGAGGGCCAGGGCAGACAGGAGACCCTGTTCCGGTGCATCCGGAGCATG
CCCAGCCACCCCGACAGAGCCTACAACAGCTGCTACAGCGCTGGCGTGTTTCACCTGCACCAG
GGCGACATCCTGAGCGTGATCATCCCCAGAGCCAGAGCCAAGCTGAACCTGTCCCCCCACGGC
ACCTTTCTGGGCTTCGTGAAGCTGTCTGGAGGCGGCTCGGATCCCGCCGAGCCCAAATCTCCT
GACAAAACTCACACATGCCCACCGTGCCCAGCACCTCCCGTGGCCGGCCCGTCAGTCTTCCTC
TTCCCCCCAAAACCCAAGGACACCCTCATGATCGCCCGGACCCCTGAGGTCACATGCGTGGTG
GTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAG
GTGTACACCCTGCCCCCATCCCGGGATGAGCTGACCAAGAACCAGGTCAGCCTGACCTGCCTG
GTCAAAGGCTTCTATCCCAGCGACATCGCCGTGGAGTGGGAGAGCAATGGGCAACCGGAGAAC
AACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAAAAAGATCCCAAATTT
TGGGTGCTGGTGGTGGTTGGTGGAGTCCTGGCTTGCTATAGCTTGCTAGTAACAGTGGCCTTT
ATTATTTTCTGGGTGAGGAGTAAGAGGAGCAGGCTCCTGCACAGTGACTACATGAACATGACT
CCCCGCCGCCCCGGGCCCACCCGCAAGCATTACCAGCCCTATGCCCCACCACGCGACTTCGCA
GCCTATCGCTCCAGGGACCAGAGGCTGCCCCCCGATGCCCACAAGCCCCCTGGGGGAGGCAGT
TTCCGGACCCCCATCCAAGAGGAGCAGGCCGACGCCCACTCCACCCTGGCCAAGATCAGAGTG
AAGTTCAGCAGGAGCGCAGACGCCCCCGCGTACCAGCAGGGCCAGAACCAGCTCTATAACGAG
CTCAATCTAGGACGAAGAGAGGAGTACGATGTTTTGGACAAGAGACGTGGCCGGGACCCTGAG
ATGGGGGGAAAGCCGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCACATGCAG
GCCCTGCCTCCTCGCTAA
(dAPRIL-CD8STK-CD28OXZ)
SEQ ID No. 16
ATGGAGACCGACACCCTGCTGCTGTGGGTGCTGCTGCTGTGGGTGCCAGGCAGCACCGGCAGC
GTGCTCCACCTGGTGCCCATCAACGCCACCAGCAAGGACGACTCTGATGTGACCGAGGTGATG
TGGCAGCCAGCCCTGAGACGGGGCAGAGGCCTGCAGGCCCAGGGCTACGGCGTGAGAATCCAG
GACGCTGGCGTGTACCTGCTGTACTCCCAGGTGCTGTTCCAGGACGTGACCTTCACAATGGGC
CAGGTGGTGAGCCGGGAGGGCCAGGGCAGACAGGAGACCCTGTTCCGGTGCATCCGGAGCATG
CCCAGCCACCCCGACAGAGCCTACAACAGCTGCTACAGCGCTGGCGTGTTTCACCTGCACCAG
GGCGACATCCTGAGCGTGATCATCCCCAGAGCCAGAGCCAAGCTGAACCTGTCCCCCCACGGC
ACCTTTCTGGGCTTCGTGAAGCTGTCTGGAGGCGGCTCGGATCCCACCACGACGCCAGCGCCG
CGACCACCAACACCGGCGCCCACCATCGCGTCGCAGCCCCTGTCCCTGCGCCCAGAGGCGTGC
CGGCCAGCGGCGGGGGGCGCAGTGCACACGAGGGGGCTGGACTTCGCCTGTGATATCTTTTGG
GTGCTGGTGGTGGTTGGTGGAGTCCTGGCTTGCTATAGCTTGCTAGTAACAGTGGCCTTTATT
ATTTTCTGGGTGAGGAGTAAGAGGAGCAGGCTCCTGCACAGTGACTACATGAACATGACTCCC
CGCCGCCCCGGGCCCACCCGCAAGCATTACCAGCCCTATGCCCCACCACGCGACTTCGCAGCC
TATCGCTCCAGGGACCAGAGGCTGCCCCCCGATGCCCACAAGCCCCCTGGGGGAGGCAGTTTC
CGGACCCCCATCCAAGAGGAGCAGGCCGACGCCCACTCCACCCTGGCCAAGATCAGAGTGAAG
TTCAGCAGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCAGAAAGATAAG
ATGGCGGAGGCCTACAGTGAGATTGGGATGAAAGGCGAGCGCCGGAGGGGCAAGGGGCACGAT
GGCCTTTACCAGGGTCTCAGTACAGCCACCAAGGACACCTACGACGCCCTTCACATGCAGGCC
CTGCCTCCTCGCTAA
(dAPRIL-HNG-CD28OXZ)
SEQ ID No. 17
ATGGAGACCGACACCCTGCTGCTGTGGGTGCTGCTGCTGTGGGTGCCAGGCAGCACCGGCAGC
GTGCTCCACCTGGTGCCCATCAACGCCACCAGCAAGGACGACTCTGATGTGACCGAGGTGATG
TGGCAGCCAGCCCTGAGACGGGGCAGAGGCCTGCAGGCCCAGGGCTACGGCGTGAGAATCCAG
GACGCTGGCGTGTACCTGCTGTACTCCCAGGTGCTGTTCCAGGACGTGACCTTCACAATGGGC
CAGGTGGTGAGCCGGGAGGGCCAGGGCAGACAGGAGACCCTGTTCCGGTGCATCCGGAGCATG
CCCAGCCACCCCGACAGAGCCTACAACAGCTGCTACAGCGCTGGCGTGTTTCACCTGCACCAG
GGCGACATCCTGAGCGTGATCATCCCCAGAGCCAGAGCCAAGCTGAACCTGTCCCCCCACGGC
ACCTTTCTGGGCTTCGTGAAGCTGTCTGGAGGCGGCTCGGATCCCGCCGAGCCCAAATCTCCT
GACAAAACTCACACATGCCCACCGTGCCCAAAAGATCCCAAATTTTGGGTGCTGGTGGTGGTT
GGTGGAGTCCTGGCTTGCTATAGCTTGCTAGTAACAGTGGCCTTTATTATTTTCTGGGTGAGG
AGTAAGAGGAGCAGGCTCCTGCACAGTGACTACATGAACATGACTCCCCGCCGCCCCGGGCCC
ACCCGCAAGCATTACCAGCCCTATGCCCCACCACGCGACTTCGCAGCCTATCGCTCCAGGGAC
CAGAGGCTGCCCCCCGATGCCCACAAGCCCCCTGGGGGAGGCAGTTTCCGGACCCCCATCCAA
GAGGAGCAGGCCGACGCCCACTCCACCCTGGCCAAGATCAGAGTGAAGTTCAGCAGGAGCGCA
GACGCCCCCGCGTACCAGCAGGGCCAGAACCAGCTCTATAACGAGCTCAATCTAGGACGAAGA
GAGGAGTACGATGTTTTGGACAAGAGACGTGGCCGGGACCCTGAGATGGGGGGAAAGCCGAGA
AGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCTCCTCGCTAA
(dAPRIL-HCH2CH3pvaa-CD28OXZ)
SEQ ID No. 18
ATGGGCACCTCCCTGCTGTGCTGGATGGCCCTGTGCCTGCTGGGAGCCGACCACGCCGACGGC
AAGCCCATTCCCAACCCCCTGCTGGGCCTGGACTCCACCTCTGGCGGAGGCGGCAGCGTGCTG
CACCTGGTGCCCATCAACGCCACCAGCAAGGACGACTCTGATGTGACCGAGGTGATGTGGCAG
CCAGCCCTGAGACGGGGCAGAGGCCTGCAGGCCCAGGGCTACGGCGTGAGAATCCAGGACGCT
GGCGTGTACCTGCTGTACTCCCAGGTGCTGTTCCAGGACGTGACCTTCACAATGGGCCAGGTG
GTGAGCCGGGAGGGCCAGGGCAGACAGGAGACCCTGTTCCGGTGCATCCGGAGCATGCCCAGC
CACCCCGACAGAGCCTACAACAGCTGCTACAGCGCTGGCGTGTTTCACCTGCACCAGGGCGAC
ATCCTGAGCGTGATCATCCCCAGAGCCAGAGCCAAGCTGAACCTGTCCCCCCACGGCACCTTT
CTGGGCTTCGTGAAGCTGTCTGGAGGCGGCTCGGATCCCGCCGAGCCCAAATCTCCTGACAAA
ACTCACACATGCCCACCGTGCCCAGCACCTCCCGTGGCCGGCCCGTCAGTCTTCCTCTTCCCC
CCAAAACCCAAGGACACCCTCATGATCGCCCGGACCCCTGAGGTCACATGCGTGGTGGTGGAC
GTGAGCCACGAAGACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGTCAGCGTCCTCACC
GTCCTGCACCAGGACTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAA
GGCTTCTATCCCAGCGACATCGCCGTGGAGTGGGAGAGCAATGGGCAACCGGAGAACAACTAC
AAGACCACGCCTCCCGTGCTGGACTCCGACGGCTCCTTCTTCCTCTACAGCAAGCTCACCGTG
GACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTAAAAAAGATCCCAAATTTTGGGTG
CTGGTGGTGGTTGGTGGAGTCCTGGCTTGCTATAGCTTGCTAGTAACAGTGGCCTTTATTATT
TTCTGGGTGAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCAGCCTAT
CGCTCCAGGGACCAGAGGCTGCCCCCCGATGCCCACAAGCCCCCTGGGGGAGGCAGTTTCCGG
ACCCCCATCCAAGAGGAGCAGGCCGACGCCCACTCCACCCTGGCCAAGATCAGAGTGAAGTTC
AGCAGGAGCGCAGACGCCCCCGCGTACCAGCAGGGCCAGAACCAGCTCTATAACGAGCTCAAT
CTAGGACGAAGAGAGGAGTACGATGTTTTGGACAAGAGACGTGGCCGGGACCCTGAGATGGGG
GGAAAGCCGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCACGATGGC
CTTTACCAGGGTCTCAGTACAGCCACCAAGGACACCTACGACGCCCTTCACATGCAGGCCCTG
CCTCCTCGCTAA
(dAPRIL-CD8STK-CD28OXZ)
SEQ ID No. 19
ATGGGCACCTCCCTGCTGTGCTGGATGGCCCTGTGCCTGCTGGGAGCCGACCACGCCGACGGC
AAGCCCATTCCCAACCCCCTGCTGGGCCTGGACTCCACCTCTGGCGGAGGCGGCAGCGTGCTG
CACCTGGTGCCCATCAACGCCACCAGCAAGGACGACTCTGATGTGACCGAGGTGATGTGGCAG
CCAGCCCTGAGACGGGGCAGAGGCCTGCAGGCCCAGGGCTACGGCGTGAGAATCCAGGACGCT
GGCGTGTACCTGCTGTACTCCCAGGTGCTGTTCCAGGACGTGACCTTCACAATGGGCCAGGTG
GTGAGCCGGGAGGGCCAGGGCAGACAGGAGACCCTGTTCCGGTGCATCCGGAGCATGCCCAGC
CACCCCGACAGAGCCTACAACAGCTGCTACAGCGCTGGCGTGTTTCACCTGCACCAGGGCGAC
ATCCTGAGCGTGATCATCCCCAGAGCCAGAGCCAAGCTGAACCTGTCCCCCCACGGCACCTTT
CTGGGCTTCGTGAAGCTGTCTGGAGGCGGCTCGGATCCCACCACGACGCCAGCGCCGCGACCA
CCAACACCGGCGCCCACCATCGCGTCGCAGCCCCTGTCCCTGCGCCCAGAGGCGTGCCGGCCA
GCGGCGGGGGGCGCAGTGCACACGAGGGGGCTGGACTTCGCCTGTGATATCTTTTGGGTGCTG
GTGGTGGTTGGTGGAGTCCTGGCTTGCTATAGCTTGCTAGTAACAGTGGCCTTTATTATTTTC
TGGGTGAGGAGTAAGAGGAGCAGGCTCCTGCACAGTGACTACATGAACATGACTCCCCGCCGC
CCCGGGCCCACCCGCAAGCATTACCAGCCCTATGCCCCACCACGCGACTTCGCAGCCTATCGC
TCCAGGGACCAGAGGCTGCCCCCCGATGCCCACAAGCCCCCTGGGGGAGGCAGTTTCCGGACC
CCCATCCAAGAGGAGCAGGCCGACGCCCACTCCACCCTGGCCAAGATCAGAGTGAAGTTCAGC
AGGAGCGCAGACGCCCCCGCGTACCAGCAGGGCCAGAACCAGCTCTATAACGAGCTCAATCTA
GGACGAAGAGAGGAGTACGATGTTTTGGACAAGAGACGTGGCCGGGACCCTGAGATGGGGGGA
AAGCCGAGAAGGAAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAGGCCCTGCCT
CCTCGCTAA
(dAPRIL-HNG-CD28OXZ)
SEQ ID No. 20
ATGGGCACCTCCCTGCTGTGCTGGATGGCCCTGTGCCTGCTGGGAGCCGACCACGCCGACGGC
AAGCCCATTCCCAACCCCCTGCTGGGCCTGGACTCCACCTCTGGCGGAGGCGGCAGCGTGCTG
CACCTGGTGCCCATCAACGCCACCAGCAAGGACGACTCTGATGTGACCGAGGTGATGTGGCAG
CCAGCCCTGAGACGGGGCAGAGGCCTGCAGGCCCAGGGCTACGGCGTGAGAATCCAGGACGCT
GGCGTGTACCTGCTGTACTCCCAGGTGCTGTTCCAGGACGTGACCTTCACAATGGGCCAGGTG
GTGAGCCGGGAGGGCCAGGGCAGACAGGAGACCCTGTTCCGGTGCATCCGGAGCATGCCCAGC
CACCCCGACAGAGCCTACAACAGCTGCTACAGCGCTGGCGTGTTTCACCTGCACCAGGGCGAC
ATCCTGAGCGTGATCATCCCCAGAGCCAGAGCCAAGCTGAACCTGTCCCCCCACGGCACCTTT
CTGGGCTTCGTGAAGCTGTCTGGAGGCGGCTCGGATCCCGCCGAGCCCAAATCTCCTGACAAA
ACTCACACATGCCCACCGTGCCCAAAAGATCCCAAATTTTGGGTGCTGGTGGTGGTTGGTGGA
GTCCTGGCTTGCTATAGCTTGCTAGTAACAGTGGCCTTTATTATTTTCTGGGTGAGGAGTAAG
AGGAGCAGGCTCCTGCACAGTGACTACATGAACATGACTCCCCGCCGCCCCGGGCCCACCCGC
AAGCATTACCAGCCCTATGCCCCACCACGCGACTTCGCAGCCTATCGCTCCAGGGACCAGAGG
CTGCCCCCCGATGCCCACAAGCCCCCTGGGGGAGGCAGTTTCCGGACCCCCATCCAAGAGGAG
CAGGCCGACGCCCACTCCACCCTGGCCAAGATCAGAGTGAAGTTCAGCAGGAGCGCAGACGCC
CCCGCGTACCAGCAGGGCCAGAACCAGCTCTATAACGAGCTCAATCTAGGACGAAGAGAGGAG
TACGATGTTTTGGACAAGAGACGTGGCCGGGACCCTGAGATGGGGGGAAAGCCGAGAAGGAAG
AACCCTCAGGAAGGCCTGTACAATGAACTGCAGAAAGATAAGATGGCGGAGGCCTACAGTGAG
ATTGGGATGAAAGGCGAGCGCCGGAGGGGCAAGGGGCACGATGGCCTTTACCAGGGTCTCAGT
ACAGCCACCAAGGACACCTACGACGCCCTTCACATGCAGGCCCTGCCTCCTCGCTAA

The nucleic acid sequence may encode the same amino acid sequence as that encoded by SEQ ID No. 15, 16, 17, 18 19 or 20 but may have a different nucleic acid sequence, due to the degeneracy of the genetic code. The nucleic acid sequence may have at least 80, 85, 90, 95, 98 or 99% identity to the sequence shown as SEQ ID No. 15, 16, 17, 18 19 or 20 provided that it encodes a CAR as defined in the first aspect of the invention.

### Vector

The present invention also provides a vector which comprises a nucleic acid sequence according to the present invention. Such a vector may be used to introduce the nucleic acid sequence into a host cell so that it expresses and produces a molecule according to the first aspect of the invention.

The vector may, for example, be a plasmid or synthetic mRNA or a viral vector, such as a retroviral vector or a lentiviral vector.

The vector may be capable of transfecting or transducing an effector cell.

### Host Cell

The invention also provides a host cell which comprises a nucleic acid according to the invention. The host cell may be capable of expressing a CAR according to the first aspect of the invention.

The host cell may be human T cell or a human NK cell.

A T-cell capable of expressing a CAR according to the invention may be made by transducing or transfecting a T cell with CAR-encoding nucleic acid.

The T-cell may be an ex vivo T cell. The T cell may be from a peripheral blood mononuclear cell (PBMC) sample. T cells may be activated and/or expanded prior to being transduced with CAR-encoding nucleic acid, for example by treatment with a anti-CD3 monoclonal antibody.

### Pharmaceutical Composition

The present invention also relates to a pharmaceutical composition containing a vector or a CAR-expressing T cell of the invention together with a pharmaceutically acceptable carrier, diluent or excipient, and optionally one or more further pharmaceutically active polypeptides and/or compounds. Such a formulation may, for example, be in a form suitable for intravenous infusion).

### Method of Treatment

T cells expressing a CAR molecule of the present invention are capable of killing cancer cells, such as multiple myeloma cells. CAR-expressing T cells may either be created ex vivo either from a patient's own peripheral blood (1^{st} party), or in the setting of a haematopoietic stem cell transplant from donor peripheral blood (2^{nd} party), or peripheral blood from an unconnected donor (3^{rd} party). Alternatively, CAR T-cells may be derived from ex-vivo differentiation of inducible progenitor cells or embryonic progenitor cells to T-cells. In these instances, CAR T-cells are generated by introducing DNA or RNA coding for the CAR by one of many means including transduction with a viral vector, transfection with DNA or RNA.

T cells expressing a CAR molecule of the present invention may be used for the treatment of a cancerous disease, in particular a plasma cell disorder or a B cell disorder which correlates with enhanced BCMA expression.

Plasma cell disorders include plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma (POEMS Syndrome) and heavy chain diseases as well as the clinically unclear monoclonal gammopathy of undetermined significance/smoldering multiple myeloma.

The disease may be multiple myeloma.

Examples for B cell disorders which correlate with elevated BCMA expression levels are CLL (chronic lymphocytic leukemia) and non-Hodgkins lymphoma (NHL). The bispecific binding agents of the invention may also be used in the therapy of autoimmune diseases like Systemic Lupus Erythematosus (SLE), multiple sclerosis (MS) and rheumatoid arthritis (RA).

The method of the present invention may be for treating a cancerous disease, in particular a plasma cell disorder or a B cell disorder which correlates with enhanced BCMA expression.

A method for the treatment of disease relates to the therapeutic use of a vector or T cell of the invention. In this respect, the vector or T cell may be administered to a subject having an existing disease or condition in order to lessen, reduce or improve at least one symptom associated with the disease and/or to slow down, reduce or block the progression of the disease. The method of the invention may cause or promote T-cell mediated killing of BCMA-expressing cells, such as plasma cells.

The invention will now be further described by way of Examples, which are meant to serve to assist one of ordinary skill in the art in carrying out the invention and are not intended in any way to limit the scope of the invention.

### EXAMPLES

### Example 1—Characterisation of BCMA as a Target for Myeloma

Primary myeloma cells were isolated by performing a CD138 immunomagnetic selection on fresh bone marrow samples from Multiple myeloma patients that were known to have frank disease. These cells were stained with the BCMA specific J6MO mAb (GSK) which was conjugated to PE. At the same time, a standard of beads with known numbers of binding sites was generated using the PE Quantibrite bead kit (Becton Dickenson) as per the manufacturer's instructions. The BCMA copy number on myeloma cells could be derived by correlating the mean-fluorescent intensity from the myeloma cells with the standard curve derived from the beads. It was found that the range of BCMA copy number on a myeloma cell surface is low: at 348.7-4268.4 BCMA copies per cell with a mean of 1181 and a median of 1084.9 (FIG. 2). This is considerably lower than e.g. CD19 and GD2, classic targets for CARs. Presence of BCMA expression on primary myeloma cells was also confirmed with the Vicky-1 antibody (Abcam Ab17323), examples of which are shown in FIG. 14.

### Example 2—Design and Construction of APRIL Based CARs

APRIL in its natural form is a secreted type II protein. The use of APRIL as a BCMA binding domain for a CAR requires conversion of this type II secreted protein to a type I membrane bound protein and for this protein to be stable and to retain binding to BCMA in this form. To generate candidate molecules, the extreme amino-terminus of APRIL was deleted to remove binding to proteoglycans. Next, a signal peptide was added to direct the nascent protein to the endoplasmic reticulum and hence the cell surface. Also, because the nature of spacer used can alter the function of a CAR, three different spacer domains were tested: an APRIL based CAR was generated comprising (i) a human IgG1 spacer altered to remove Fc binding motifs; (ii) a CD8 stalk; and (iii) the IgG1 hinge alone (cartoon in FIGS. 4A-4C and amino acid sequences in FIGS. 5A-5C, and also amino acid sequences in FIG. 19 which differ from the sequences in FIGS. 5A-5C by having a different signal peptide and the V5 epitope tag). These CARs were expressed in a bicistronic retroviral vector (FIG. 6A) so that a marker protein-truncated CD34 could be co-expressed as a convenient marker gene.

### Example 3—Expression and Function of APRIL Based CARs

The aim of this study was to test whether the APRIL based CARs which had been constructed were expressed on the cell surface and whether APRIL had folded to form the native protein. T-cells were transduced with these different CAR constructs and stained using a commercially available anti-APRIL mAb, along with staining for the marker gene and analysed by flow-cytometry. The results of this experiment are shown in FIG. 6B where APRIL binding is plotting against marker gene fluorescence. These data show that in this format, the APRIL based CARs are expressed on the cell surface and APRIL folds sufficiently to be recognized by an anti-APRIL mAb.

Next, it was determined whether APRIL in this format could recognize BCMA and TACI. Recombinant BCMA and TACI were generated as fusions with mouse IgG2a-Fc. These recombinant proteins were incubated with the transduced T-cells. After this, the cells were washed and stained with an anti-mouse fluorophore conjugated antibody and an antibody to detect the marker gene conjugated to a different fluorophore. The cells were analysed by flow cytometry and the results are presented in FIG. 6C. The different CARS were able to bind both BCMA and TACI. Surprisingly, the CARs were better able to bind BCMA than TACI. Also, surprisingly CARs with a CD8 stalk or IgG1 hinge spacer were better able to bind BCMA and TACI than CAR with an Fc spacer.

### Example 4—APRIL Based Chimeric Antigen Receptors are Active Against BCMA Expressing Cells

T-cells from normal donors were transduced with the different APRIL CARs and tested against SupT1 cells either wild-type, or engineered to express BCMA and TACI. Several different assays were used to determine function. A classical chromium release assay was performed. Here, the target cells (the SupT1 cells) were labelled with ⁵¹Cr and mixed with effectors (the transduced T-cells) at different ratio. Lysis of target cells was determined by counting ⁵¹Cr in the co-culture supernatant (FIG. 6A shows the cumulative data, example data from a single assay with different effector: target ratios is shown in FIG. 12).

In addition, supernatant from T-cells cultured 1:1 with SupT1 cells was assayed by ELISA for Interferon-gamma (FIG. 6B shows cumulative data, example data from a single assay is shown in FIG. 13). Measurement of T-cell expansion after one week of co-culture with SupT1 cells was also performed (FIG. 6C). T-cells were counted by flow-cytometry calibrated with counting beads. These experimental data show that APRIL based CARs can kill BCMA expressing targets. Further, these data show that CARs based on the CD8 stalk or IgG1 hinge performed better than the Fc-pvaa based CAR.

### Example 5—APRIL Based CARs are Able to Kill Primary Myeloma Cells

The above data are encouraging since they demonstrate that it in principle, it is possible to make an APRIL based CAR. However, since most primary myeloma cells express a low number of BCMA molecules on their surface, it was investigated whether such an APRIL based CAR would cause killing of primary myeloma cells, particularly in cases with low-density expression. Three cases were selected which represented the range of BCMA expression described in FIG. 2: the first had dim expression (lower than mean); the second case had intermediate expression (approximately mean expression) and the third had bright (above mean expression). FIG. 8 shows a histogram of BCMA staining against isotype control for all three cases on the left to illustrate BCMA expression. Since when comparing APRIL based CARs with different spacers it had been determined that CARs with CD8 stalk spacer and IgG1 hinge spacer performed better than the Fc-pvaa spacered CAR, in this assay, only the CD8 stalk and hinge APRIL CARs were tested. On the left, survival of myeloma cells compared with starting numbers is shown at day 3 and day 6 after a 1:1 co-culture of myeloma cells and CAR T-cells. By day 6, >95% of the myeloma cells were eliminated, including those with dim BCMA expression. Dim BCMA expressing myeloma cells can be targeted by the APRIL CARs albeit with a slower tempo of killing than higher expressers.

### Example 6—Secreted and Truncated APRIL Fused to an Fc Spacer Recognizes BCMA and TACI

In order to investigate whether truncated APRIL in a CAR format (i.e. fused to a transmembrane domain and anchored to a cell membrane) could bind BCMA and TACI, a basic CAR was engineered in frame with the self-cleaving foot and mouth disease 2A peptide with truncated CD34, as a convenient marker gene. A stable SUPT1 cell line was established which expresses this construct. Secreted truncated BCMA and TACI fused to human (and other species, not shown) Ig Fc domain was also generated and recombinant protein produced. It was shown that both BCMA-Fc and TACI-Fc bind the engineered SUPT1 cell line. Only cells expressing the CD34 marker gene were found to bind BCMA-Fc and TACI-Fc (FIG. 9).

### Example 7—APRIL Based Chimeric Antigen Receptors are Stably Expressed on the Surface of T-Cells

The CAR spacer domain can alter sensitivity and specificity. Three versions of an APRIL-based CAR were generated with three spacer domains: (i) a human IgG1 spacer altered to remove Fc binding motifs; (ii) a CD8 stalk; and (iii) the IgG1 hinge alone (FIG. 10B). Primary human T-cells were transduced with these different CARS and stained using a commercially available anti-APRIL mAb (FIG. 11).

### Example 8—APRIL Based Chimeric Antigen Receptors are Active Against Cognate Target Expressing Cells

T-cells from normal donors were transduced with the different APRIL CARs and tested against SupT1 cells either wild-type, or engineered to express BCMA and TACI. Several different assays were used to determine function. A classical chromium release assay was performed. Here, the target cells (the SupT1 cells) were labelled with ⁵¹Cr and mixed with effectors (the transduced T-cells) at different ratio. Lysis of target cells was determined by counting ⁵¹Cr in the co-culture supernatant (FIG. 12).

In addition, supernatant from T-cells cultured 1:1 with SupT1 cells was assayed by ELISA for Interferon-gamma (FIG. 13).

Measurement of T-cell expansion after one week of co-culture with SupT1 cells was also performed. T-cells were counted by flow-cytometry calibrated with counting beads. Initial data (not shown) appears to indicate that the CD8 stalk based construct results in more T-cell proliferation than the other constructs.

### Example 9—Demonstration of In Vivo Function of APRIL CAR T-Cells

In order to demonstrate APRIL CAR T-cell function in vivo, APRIL CAR T-cells were tested in a human/mouse chimeric model. MM1·s (ATCC CRL-2974) is a human myeloma cell line which expresses intermediate levels of BCMA. The inventors engineered this cell line to express firefly Luciferase to derive the cell-line MM1·s·FLuc.

NOD scid gamma (NSG: NOD·Cg-Prkdo^{scid} II2rgtm1^{Wjl/SzJ}) mice are profoundly immunosuppressed mice capable of engrafting several human cell lines and human peripheral blood lymphocytes. Three month old female NSG mice received 1×10⁷ MM1·s·FLuc cells vial tail-vein injection without any preparative therapy. Engraftment was determined by serial bioluminescence imaging (FIG. 16). Robust and increasing intramedullary engraftment was observed in all mice. At day 13, 5×10⁶ APRIL-HNG-CD28OXZ CAR T-cells were administered via tail vein injection. Serial bioluminescence was performed which showed rapid decrease in burden of MM1·s (FIG. 16) in all treated mice to a complete remission. This response to CAR therapy was confirmed by flow-cytometry and immunohistochemistry.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1. A chimeric antigen receptor (CAR) comprising:
(i) a B cell maturation antigen (BCMA)-binding domain which comprises at least part of a proliferation-inducing ligand (APRIL),
(ii) a spacer domain,
(iii) a transmembrane domain, and
(iv) (iii) an intracellular T cell signaling domain.

2. A CAR according to claim 1, wherein the BCMA-binding domain comprises a truncated APRIL which comprises the BCMA binding site but lacks the amino terminal portion of APRIL responsible for proteoglycan binding.

3. A CAR according to claim 2, which comprises the sequence shown as SEQ ID No. 14 or a variant thereof having at least 80% sequence identity which binds BCMA.

4. A CAR according to claim 1, wherein the transmembrane and intracellular T-cell signalling domain comprise the sequence shown as SEQ ID No. 7 or a variant thereof having at least 80% sequence identity.

5. A CAR according to claim 1, wherein the spacer comprises one of the following: a human IgG1 spacer, an IgG1 hinge, or a CD8 stalk.

6. A CAR according to claim 5, wherein the spacer comprises a CD8 stalk.

7. A CAR according to claim 1, which comprises the sequence shown as SEQ ID No. 1, 2, 3, 4, 5 or 6 or a variant thereof which has at least 80% sequence identity but retains the capacity to i) bind BCMA and ii) induce T cell signalling.

8. A nucleic acid sequence which encodes a CAR according to claim 1.

9. A nucleic acid sequence according to claim 8 which comprises the sequence shown as SEQ ID No 15, 16, 17, 18, 19 or 20 or a variant thereof having at least 80% sequence identity.

10. A vector which comprises a nucleic acid sequence according to claim 8.

11. A T cell or NK cell which expresses a CAR according to claim 1.

12. A method for making a T cell or NK cell according to claim 11, which comprises the step of introducing into a T cell or NK cell a nucleic acid which encodes a CAR comprising:
(i) a B cell maturation antigen (BCMA)-binding domain which comprises at least part of a proliferation-inducing ligand (APRIL),
(ii) a spacer domain,
(iii) a transmembrane domain, and
(iv) an intracellular T cell signaling domain.

13. A pharmaceutical composition which comprises a vector according to claim 10, together with a pharmaceutically acceptable carrier, diluent or excipient.

14. A method for treating a plasma cell disorder which comprises the step of administering a vector according to claim 10 to a subject.

15. A method according to claim 14, wherein the plasma cell disorder is selected from plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance and smoldering multiple myeloma.

16. A method according to claim 15, wherein the plasma cell disorder is multiple myeloma.

17-18. (canceled)

19. A pharmaceutical composition which comprises a T cell/NK cell according to claim 11, together with a pharmaceutically acceptable carrier, diluent or excipient.

20. A method for treating a plasma cell disorder which comprises the step of administering a T cell or NK cell according to claim 11 to a subject.

21. A method according to claim 20, wherein the plasma cell disorder is selected from plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance and smoldering multiple myeloma.

22. A method according to claim 20, wherein the plasma cell disorder is multiple myeloma.


SEQUENCE LISTING (21 sequences)

SEQ ID NO: 1 - protein, 614 aa, synthetic construct
  REGION 1..614: note = Chimeric antigen receptor (CAR) dAPRIL-HCH2CH3pvaa-CD28OXZ
METDTLLLWVLLLWVPGSTGSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 424 aa, synthetic construct
  REGION 1..424: note = Chimeric antigen receptor (CAR) dAPRIL-CD8STK-CD28OXZ
METDTLLLWVLLLWVPGSTGSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 398 aa, synthetic construct
  REGION 1..398: note = Chimeric antigen receptor (CAR) dAPRIL-HNG-CD28OXZ
METDTLLLWVLLLWVPGSTGSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPAEPKSPDKTHTCPPCPKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 633 aa, synthetic construct [sequence omitted]

SEQ ID NO: 5 - protein, 443 aa, synthetic construct
  REGION 1..443: note = Chimeric antigen receptor (CAR) dAPRIL-CD8STK-CD28OXZ
MGTSLLCWMALCLLGADHADGKPIPNPLLGLDSTSGGGGSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 417 aa, synthetic construct
  REGION 1..417: note = Chimeric antigen receptor (CAR) dAPRIL-HNG-CD28OXZ
MGTSLLCWMALCLLGADHADGKPIPNPLLGLDSTSGGGGSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPAEPKSPDKTHTCPPCPKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 216 aa, synthetic construct
  REGION 1..216: note = Transmembrane and intracellular T-cell signalling domain (endodomain)
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 21 aa, synthetic construct
  REGION 1..21: note = Signal peptide
MGTSLLCWMALCLLGADHADG

SEQ ID NO: 9 - protein, 20 aa, synthetic construct
  REGION 1..20: note = Signal peptide
METDTLLLWVLLLWVPGSTG

SEQ ID NO: 10 - protein, 234 aa, synthetic construct
  REGION 1..234: note = Spacer (hinge-CH2CH3 of human IgG1)
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 11 - protein, 46 aa, synthetic construct
  REGION 1..46: note = Spacer (human CD8 stalk)
TTTPAPRPPTPAPTIASQPLSLRPEACRPAAGGAVHTRGLDFACDI

SEQ ID NO: 12 - protein, 20 aa, synthetic construct
  REGION 1..20: note = Spacer (human IgG1 hinge)
AEPKSPDKTHTCPPCPKDPK

SEQ ID NO: 13 - protein, 250 aa, Homo sapiens [sequence omitted]

SEQ ID NO: 14 - protein, 134 aa, Homo sapiens
VLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 15 - DNA, 1845 bp, synthetic construct
  misc_feature 1..1845: note = Nucleic acid sequences coding for a CAR (dAPRIL-HCH2CH3pvaa-CD28OXZ)
  source 1..1845: mol_type = other DNA
atggagaccgacaccctgctgctgtgggtgctgctgctgtgggtgccaggcagcaccggcagcgtgctccacctggtgcccatcaacgccaccagcaaggacgactctgatgtgaccgaggtgatgtggcagccagccctgagacggggcagaggcctgcaggcccagggctacggcgtgagaatccaggacgctggcgtgtacctgctgtactcccaggtgctgttccaggacgtgaccttcacaatgggccaggtggtgagccgggagggccagggcagacaggagaccctgttccggtgcatccggagcatgcccagccaccccgacagagcctacaacagctgctacagcgctggcgtgtttcacctgcaccagggcgacatcctgagcgtgatcatccccagagccagagccaagctgaacctgtccccccacggcacctttctgggcttcgtgaagctgtctggaggcggctcggatcccgccgagcccaaatctcctgacaaaactcacacatgcccaccgtgcccagcacctcccgtggccggcccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 16 - DNA, 1275 bp, synthetic construct
  misc_feature 1..1275: note = Nucleic acid sequences coding for a CAR (dAPRIL-CD8STK-CD28OXZ)
  source 1..1275: mol_type = other DNA
atggagaccgacaccctgctgctgtgggtgctgctgctgtgggtgccaggcagcaccggcagcgtgctccacctggtgcccatcaacgccaccagcaaggacgactctgatgtgaccgaggtgatgtggcagccagccctgagacggggcagaggcctgcaggcccagggctacggcgtgagaatccaggacgctggcgtgtacctgctgtactcccaggtgctgttccaggacgtgaccttcacaatgggccaggtggtgagccgggagggccagggcagacaggagaccctgttccggtgcatccggagcatgcccagccaccccgacagagcctacaacagctgctacagcgctggcgtgtttcacctgcaccagggcgacatcctgagcgtgatcatccccagagccagagccaagctgaacctgtccccccacggcacctttctgggcttcgtgaagctgtctggaggcggctcggatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 17 - DNA, 1197 bp, synthetic construct
  misc_feature 1..1197: note = Nucleic acid sequences coding for a CAR (dAPRIL-HNG-CD28OXZ)
  source 1..1197: mol_type = other DNA
atggagaccgacaccctgctgctgtgggtgctgctgctgtgggtgccaggcagcaccggcagcgtgctccacctggtgcccatcaacgccaccagcaaggacgactctgatgtgaccgaggtgatgtggcagccagccctgagacggggcagaggcctgcaggcccagggctacggcgtgagaatccaggacgctggcgtgtacctgctgtactcccaggtgctgttccaggacgtgaccttcacaatgggccaggtggtgagccgggagggccagggcagacaggagaccctgttccggtgcatccggagcatgcccagccaccccgacagagcctacaacagctgctacagcgctggcgtgtttcacctgcaccagggcgacatcctgagcgtgatcatccccagagccagagccaagctgaacctgtccccccacggcacctttctgggcttcgtgaagctgtctggaggcggctcggatcccgccgagcccaaatctcctgacaaaactcacacatgcccaccgtgcccaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 18 - DNA, 1902 bp, synthetic construct
  misc_feature 1..1902: note = Nucleic acid sequences coding for a CAR (dAPRIL-HCH2CH3pvaa-CD28OXZ)
  source 1..1902: mol_type = other DNA
atgggcacctccctgctgtgctggatggccctgtgcctgctgggagccgaccacgccgacggcaagcccattcccaaccccctgctgggcctggactccacctctggcggaggcggcagcgtgctgcacctggtgcccatcaacgccaccagcaaggacgactctgatgtgaccgaggtgatgtggcagccagccctgagacggggcagaggcctgcaggcccagggctacggcgtgagaatccaggacgctggcgtgtacctgctgtactcccaggtgctgttccaggacgtgaccttcacaatgggccaggtggtgagccgggagggccagggcagacaggagaccctgttccggtgcatccggagcatgcccagccaccccgacagagcctacaacagctgctacagcgctggcgtgtttcacctgcaccagggcgacatcctgagcgtgatcatccccagagccagagccaagctgaacctgtccccccacggcacctttctgggcttcgtgaagctgtctggaggcggctcggatcccgccgagcccaaatctcctgacaaaactcacacatgcccaccgtgcccagcacctcccgtggccggcccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 19 - DNA, 1332 bp, synthetic construct
  misc_feature 1..1332: note = Nucleic acid sequences coding for a CAR (dAPRIL-CD8STK-CD28OXZ)
  source 1..1332: mol_type = other DNA
atgggcacctccctgctgtgctggatggccctgtgcctgctgggagccgaccacgccgacggcaagcccattcccaaccccctgctgggcctggactccacctctggcggaggcggcagcgtgctgcacctggtgcccatcaacgccaccagcaaggacgactctgatgtgaccgaggtgatgtggcagccagccctgagacggggcagaggcctgcaggcccagggctacggcgtgagaatccaggacgctggcgtgtacctgctgtactcccaggtgctgttccaggacgtgaccttcacaatgggccaggtggtgagccgggagggccagggcagacaggagaccctgttccggtgcatccggagcatgcccagccaccccgacagagcctacaacagctgctacagcgctggcgtgtttcacctgcaccagggcgacatcctgagcgtgatcatccccagagccagagccaagctgaacctgtccccccacggcacctttctgggcttcgtgaagctgtctggaggcggctcggatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 20 - DNA, 1254 bp, synthetic construct
  misc_feature 1..1254: note = Nucleic acid sequences coding for a CAR (dAPRIL-HNG-CD28OXZ)
  source 1..1254: mol_type = other DNA
atgggcacctccctgctgtgctggatggccctgtgcctgctgggagccgaccacgccgacggcaagcccattcccaaccccctgctgggcctggactccacctctggcggaggcggcagcgtgctgcacctggtgcccatcaacgccaccagcaaggacgactctgatgtgaccgaggtgatgtggcagccagccctgagacggggcagaggcctgcaggcccagggctacggcgtgagaatccaggacgctggcgtgtacctgctgtactcccaggtgctgttccaggacgtgaccttcacaatgggccaggtggtgagccgggagggccagggcagacaggagaccctgttccggtgcatccggagcatgcccagccaccccgacagagcctacaacagctgctacagcgctggcgtgtttcacctgcaccagggcgacatcctgagcgtgatcatccccagagccagagccaagctgaacctgtccccccacggcacctttctgggcttcgtgaagctgtctggaggcggctcggatcccgccgagcccaaatctcctgacaaaactcacacatgcccaccgtgcccaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 21 - protein, 6 aa, synthetic construct
  REGION 1..6: note = Furin cleavage site
KQKKQK